(19) United States
(12) Patent Application Publication
(10) Pub. No.: US 2005/0026966 A1
(43) Pub. Date: Feb. 3, 2005

(54) Process and chiral amine intermediates useful for preparation of antiproliferative 2,4-diaminothiazole amide compounds

(75) Inventors: David John Kucera, Del Mar, CA (US); Brigitte Leigh Yvon, Encinitas, CA (US)

Correspondence Address: AGOURON PHARMACEUTICALS, INC., 10350 NORTH TORREY PINES ROAD, LA JOLLA, CA, 92037, US

(73) Assignee: Agouron Pharmaceuticals, Inc.

(21) Appl. No.: 10/631,358

(22) Filed: Jul. 30, 2003

(51) Int. Cl. C12Q 1/68; C07D 213/54; A61K 31/44; A61K 31/40; C07D 207/46

(52) U.S. Cl. 514/357; 514/553; 514/408; 546/329; 548/567; 564/414

(57) ABSTRACT

This invention relates to a novel process for preparing D-alanine derivatives and bis-toluenesulfonic acid salts thereof useful as intermediates in the preparation of 2,4-diaminothiazole amide compounds and pharmaceutical compositions containing such compounds. The invention also relates to such compounds and compositions that demonstrate antiproliferative activity such as antitumor activity. These compounds and compositions are also useful for treating various diseases and disorders associated with uncontrolled or unwanted cell proliferation and for inhibiting protein kinases.

### FIELD OF THE INVENTION

This invention relates to a novel process for preparing D-alanine derivatives and bis-toluenesulfonic acid salts thereof useful as intermediates in the preparation of 2,4-diaminothiazole amide compounds and pharmaceutical compositions containing such compounds. The invention also relates to such 2,4-diaminothiazole amide compounds and compositions that demonstrate anti-proliferative activity such as antitumor activity. These 2,4-diaminothiazole amide compounds and compositions are also useful for treating various diseases and disorders associated with uncontrolled or unwanted cell proliferation and for inhibiting protein kinases.

### BACKGROUND OF THE INVENTION

Cell proliferation occurs in response to various stimuli and may stem from de-regulation of the cell division cycle (or cell cycle), the process by which cells multiply and divide. Hyperproliferative disease states, including cancer, are characterized by cells rampantly winding through the cell cycle with uncontrolled vigor due to, for example, damage to the genes that directly or indirectly regulate progression through the cycle. Thus, agents that modulate the cell cycle, and thus hyperproliferation, could be used to treat various disease states associated with uncontrolled or unwanted cell proliferation. In addition to cancer chemotherapeutic agents, cell cycle inhibitors are also proposed as antiparasitics (Gray et al., Curr. Med. Chem., 6, 859-875 (1999)) and recently demonstrated as potential antivirals. See, e.g., Yang et al., Nature (London), 414, 317-322 (2001); Nguyen et al., Nature (London), 414, 322-325 (2001). Moreover, the applicability of antiproliferative agents may be expanded to treating cardiovascular maladies such as artherosclerosis or restenosis (Fishbein et al., Drug Dev. Res., 50, 487-496 (2000)), and states of inflammation, such as psoriasis and arthritis. Taniguchi et al., Nat Med., 5, 760-767 (1999).

Among the candidate regulatory proteins, protein kinases are a family of enzymes that catalyze phosphorylation of the hydroxyl group of specific tyrosine, serine or threonine residues in proteins. Typically, such phosphorylation dramatically perturbs the function of the protein, and thus protein kinases are pivotal in the regulation of a wide variety of cellular processes. It is believed that as inhibitors of protein kinases, such as, for example, cyclin dependent kinases (“CDK”), inventive agents can modulate the level of cellular RNA and DNA synthesis and therefore are expected to be useful in the treatment of viral infections such as HIV, human papilloma virus, herpesvirus, Epstein-Barr virus, adenovirus, Sindbis virus, poxvirus and the like. See, e.g., Schang et al., J. Virol., 74, 2107-2120 (2000). CDK complexes are formed through association of a regulatory cyclin subunit (e.g., cyclin A, B1, B2, D1, D2, D3, and E) and a catalytic kinase subunit (e.g., CDK1, CDK2, CDK4, CDK5, and CDK6).

In addition to the protein kinases identified above, many other protein kinases have been considered to be therapeutic targets, and numerous publications disclose inhibitors of kinase activity, as reviewed in the following: Rao et al., Targets Cancer Chemothe., 145-178 (2002); Garcia-Echeverria et al., Med. Res. Rev., 20, 28-57 (2000); and Toledo et al., Curr. Med. Chem., 6, 775-805 (1999).

A number of references disclose thiazole compounds and the preparation of such compounds, including International Publication Nos. WO 99/21845 and WO 00/75120, and U.S. Patent Publication No. 2002/0025976, wherein 2,4-diaminothiazoles are used as CDK or kinase inhibitors. After an early report of 2,4-diaminothiazoles in Gewald et al., J. Prakt. Chem., 35, 97-104 (1967), subsequent modified preparations were seen in Rajasekharan et al., Synthesis, 353-355 (1986), Jenardanan et al., Syn. Comm., 27, 3457-3462 (1997), and Binu et al., Org. Prep. Proced. Intl., 30, 93-96 (1998). Yet another extension of the methodology recently appeared in Devi et al., Syn. Comm., 32,1523-1528 (2002), which alluded to the preparation of a combinatorial library of 2,4-diaminothiazoles. This was realized from another recent modification from Masquelin et al., Tetrahedron, 57,153-156 (2001), which was adapted to solid support in Baer et al., J. Comb. Chem., 3,16-19 (2001).

Aminothiazoles were also reported in U.S. Pat. No. 6,262,096, International Publication Nos. WO 01/44241, WO 01/44242, and aminobenzothiazoles in WO 99/24035. International Publication No. WO 00/17175 describes other aminothiazoles used as p38 mitogen-activated protein (MAP) kinase inhibitors, and WO 00/26202, WO 00/26203, and U.S. Pat. No. 6,114,365 describe aminothiazoles and ureidothiazoles used as anti-tumor agents.

The preparation of certain 2,4-diaminothiazole benzamide compounds that utilize certain D-alanine derivatives and their hydrochloride salts, such as (R)-N, N-dimethyl-propane-1,2-diamine dihydrochloride salt, has been described in U.S. patent application Ser. No. 10/190,219 and U.S. Provisional Application No. 60/402,408. Isogai et al., J. Chem. Soc. Perkin Trans., 1, 1405-1411 (1984) describe a three-step preparation of L-alanine derivatives and their hydrochloride salts. Grudzinski et al., Acta Pol. Pharm., 33, 571-576 (1976) disclose the preparation of a D,L-1-dimethylamino-2-aminopropane toluenesulfonic acid salt.

However, there is still a need to discover improved, more efficient, processes and novel intermediates for use in the synthesis of 2,4-diaminothiazole amide compounds with antiproliferative activity. In particular, there is a need to for improved methods for synthesizing the compounds of the general formulae I, V, and VI.

### SUMMARY OF THE INVENTION

This invention relates to an efficient four-step method for preparing novel D-alanine derivatives and their non-hygroscopic bis-toluenesulfonic acid salts. These easy-to-handle non-hygroscopic bis-toluenesulfonic acid salts of D-alanine derivatives are key intermediates in the synthesis of 2,4-diaminothiazole amide compounds, which are used to treat various diseases and disorders associated with uncontrolled or unwanted cell proliferation and for inhibiting protein kinases.

In one general aspect, the invention provides a process for preparing a compound of formula I 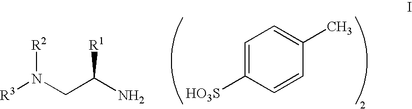
wherein:
R¹, R², and R³are independently, H, C₁-C₆ alkyl, 2-10 membered heteroalkyl, —(CR¹³R¹⁴)ₜ(C₆-C₁₀ aryl), —(CR¹³R¹⁴)ₜ(C₃-C₁₀ cycloalkyl), —(CR¹³R¹⁴)(C₆-C₁₀ heterocyclic), wherein t is an integer from 0 to 5; 1 or 2 ring carbon atoms of the cycloalkyl or heterocyclic group are optionally substituted with an oxo (═O) moiety; each R¹³ and R¹⁴ is independently H, C₁-C₆ alkyl, or 2-10 membered heteroalkyl, and wherein any of R¹, R² or R³ may be optionally substituted with one or more substituents independently selected from halo, —OH, —CN, —SR¹⁵, —NO₂, C₁-C₆ alkyl, C₂-C₆ alkenyl, C₂-C₆ alkynyl, 2-10 membered heteroalkyl, —COR¹⁵, or COOR¹⁵ wherein R¹⁵ is H, C₁-C₆ alkyl, or 2-10 membered heteroalkyl;
   comprising the steps of:

(a) coupling a compound of formula II 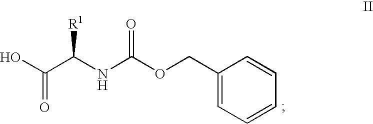
with an amine (R²)(R³)NH to form a compound of formula III

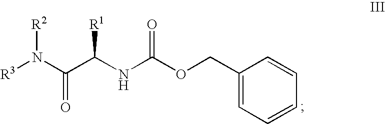

(b) deprotecting the compound of formula III to form the free amine compound of formula IV

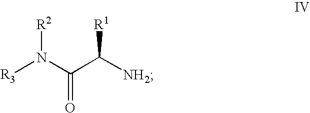

(c) reducing the free amine compound of formula IV to form a compound of formula V

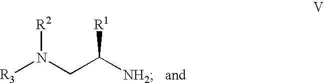

(d) treating the compound of formula V with p-toluenesulfonic acid to form the bis-toluenesulfonic acid salt compound of formula I;

wherein steps (b) and (c) can be reversed.

In one embodiment, R¹, R², and R³ are independently a C₁-C₆ alkyl, —(CR¹³R¹⁴)ₜ(C₆-C₁₀ aryl), —(CR¹³R¹⁴)(C₆-C₁₀ heterocyclic), unsubstituted or substituted with one or more substituents independently selected from the group consisting of C₁-C₆ alkyl, and —O-alkyl. Preferably, R¹, R², and R³ are independently a C₁-C₆ alkyl group, unsubstituted or substituted with one or more substituents independently selected from the group consisting of C₁-C₆ alkyl and —O-alkyl. More preferably, R¹, R², and R³ are independently an unsubstituted C₁₋₃ alkyl group. More preferably, R¹, R², and R³ are each —CH₃.

In another embodiment, steps (b) through (d) are carried out without using water as a solvent or an extraction agent.

In another embodiment, steps (c) and (d) are carried out without using water as a solvent or an extraction agent.

In another embodiment, step (d) is carried out in the absence of water.

In another embodiment, step (b) is carried out in the presence of hydrogen gas, a solvent, and a catalytic amount of metal catalyst, at a temperature from about 0° C. to about 100° C.

In another embodiment, step (c) is carried out in the presence of a hydride source and a solvent at a temperature of from about 0° C. to about 100° C. Preferably, step (c) is carried out in the presence of lithium aluminum hydride in tetrahydrofuran at a temperature of from about 20° C. to about 70° C.

In another embodiment, step (d) is carried out in the presence of tetrahydrofuran at a temperature from about 0° C. to about 70° C.

In another embodiment, step (d) is carried out in the absence of an extraction or chromatography purification of the bis-toluenesulfonic acid salt compound of formula I.

In another embodiment, steps (a) through (d) result in an overall stoichiometric yield of greater than 50% yield of the formula I compound. Preferably, steps (a) through (d) result in an overall stoichiometric yield of greater than 70% yield of the formula I compound.

In another aspect, the invention provides a process for preparing a compound of formula VI 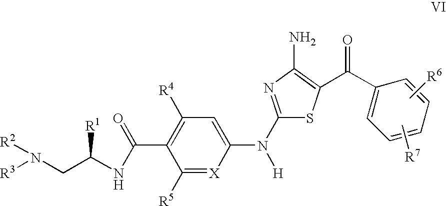
wherein:
R¹, R², and R³are independently H, C₁-C₆ alkyl, 2-10 membered heteroalkyl, —(CR¹³R¹⁴)ₜ(C₆-C₁₀ aryl), —(CR¹³R¹⁴)ₜ(C₃-C₁₀ cycloalkyl), —(CR¹³R¹⁴)(C₆-C₁₀ heterocyclic), wherein t is an integer from 0 to 5; 1 or 2 ring carbon atoms of the cycloalkyl or heterocyclic group are optionally substituted with an oxo (═O) moiety; each R¹³ and R¹⁴ is independently H, C₁-C₆ alkyl, or 2-10 membered heteroalkyl, and wherein any of R¹, R² or R³ may be optionally substituted with one or more substituents independently selected from halo, —OH, —CN, —SR¹⁵, —NO₂, C₁-C₆ alkyl, C₂-C₆ alkenyl, C₂-C₆ alkynyl, 2-10 membered heteroalkyl, —COR¹⁵, or COOR¹⁵ wherein R¹⁵ is H, C₁-C₆ alkyl, or 2-10 membered heteroalkyl;
R⁴ and R⁵ are independently H, halo, C₁-C₂ alkyl, —OCH₃, —OH, —NH₂, —NHCH₃, —N(CH₃)₂, —NO₂, —SH, —SCH₃, —S(O)CH₃, —SO ₂CH₃,P(CH₃)₂, or PO₃H₂;
R⁶ and R⁷ are independently H, halo, methoxyl, or C₁-C₂ alkyl; and
X is —C— or —N—;
comprising the steps of:

(a) coupling a compound of formula II 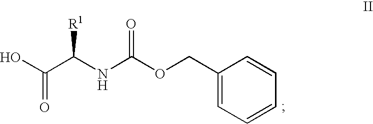
with an amine (R²)(R³)NH to form a compound of formula III

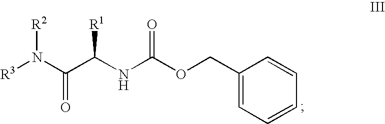

(b) deprotecting the compound of formula III to form the free amine compound of formula IV

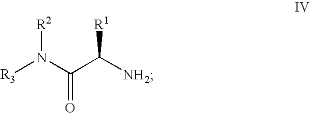

(c) reducing the free amine compound of formula IV to form a compound of formula V

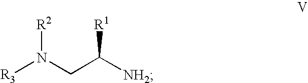

(d) treating the compound of formula V with p-toluenesulfonic acid hydrate to form the bis-toluenesulfonic acid salt compound of formula I

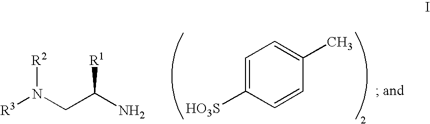

(e) coupling the bis-toluenesulfonic acid salt compound of formula I with a compound of formula VII 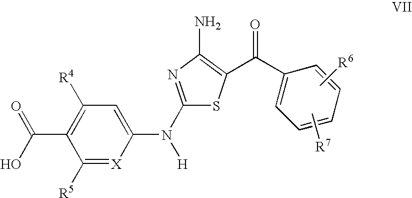
to form the compound of formula VI;

wherein steps (b) and (c) can be reversed.
In one embodiment, R¹, R², and R³ are independently a C₁-C₆ alkyl, 2-10 membered heteroalkyl, —(CR¹³R¹⁴)ₜ(C₆-C₁₀ aryl), —(CR¹³R¹⁴)(C₆-C₁₀ heterocyclic), wherein t is an integer from 0 to 5; 1 or 2 ring carbon atoms of the cycloalkyl or heterocyclic group are optionally substituted with an oxo (═O) moiety; each R¹³ and R¹⁴ is independently H, C₁-C₆ alkyl, or 2-10 membered heteroalkyl, and wherein any of R¹, R² or R³ may be optionally substituted with one or more substituents independently selected from halo, —OH, —CN, —SR¹⁵, —NO₂, C₁-C₆ alkyl, C₂-C₆ alkenyl, C₂-C₆ alkynyl, 2-10 membered heteroalkyl, —COR¹⁵, or COOR¹⁵ wherein R¹⁵ is H, C₁-C₆ alkyl, or 2-10 membered heteroalkyl; R⁴ and R⁵ are independently H, halo, C₁₋₂ alkyl, —OCH₃, —OH, —NH₂, —NHCH₃, —N(CH₃)₂, —NO₂, —SH, —SCH₃, —S(O)CH₃, —SO₂CH₃,P(CH₃)₂, or PO₃H₂; R⁶ and R⁷ are independently H, halo, methoxyl, or C₁-C₂ alkyl; and X is —C— or —N—. Preferably, R¹, R², and R³ are independently a C₁-C₆ alkyl group, unsubstituted or substituted with one or more substituents independently selected from the group consisting of C₁-C₃ alkyl and —O-alkyl; R⁴ and R⁵ are independently H, halo, C₁-C₂ alkyl, —OCH₃, —OH; R⁶ and R⁷ are independently H, halo, methoxyl, or C₁-C₂ alkyl; and X is —C— or —N—. More preferably, R¹, R², and R³ are independently an unsubstituted C₁-C₃ alkyl group; R⁴ and R⁵ are independently H, halo, C₁-C₂ alkyl; R⁶ and R⁷ are independently H, halo, methoxyl, or C₁-C₂ alkyl; and X is —C— or —N—. More preferably R¹, R², and R³are each —CH₃; R⁴ and R⁵ are independently H or C₁-C₂ alkyl; R⁶ and R⁷ are independently H, halo, or C₁-C₂ alkyl; and X is —C— or —N—. More preferably, compounds of formula VI are selected from

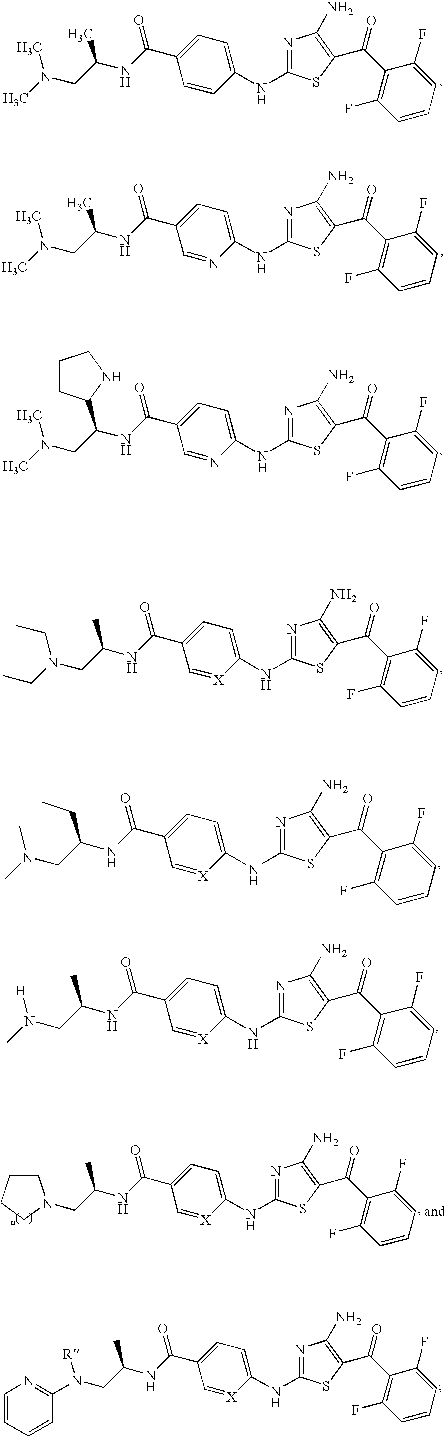

wherein n is 1 or 2 and R″ is H, —CH₃, or —CH₂CH₃.

In another embodiment, steps (b) through (d) are carried out without using water as a solvent or an extraction agent.

In another embodiment, steps (c) and (d) are carried out without using water as a solvent or an extraction agent.

In another embodiment, step (d) is carried out in the absence of water.

In another embodiment, step (b) is carried out in the presence of hydrogen gas, a solvent, and a catalytic amount of a metal catalyst, at a temperature from about 0° C. to about 100° C.

In another embodiment step (c) is carried out in the presence of a hydride source and a solvent at a temperature of from about 0° C. to about 100° C. Preferably, step (c) is carried out in the presence of lithium aluminum hydride in tetrahydrofuran at a temperature of from about 20° C. to about 70° C.

In another embodiment, step (d) is carried out in the presence of tetrahydrofuran at a temperature from about 0° C. to about 70° C. Preferably, step (d) is carried out in the absence of an extraction or chromatography purification of the bis-toluenesulfonic acid salt compound of formula I.

In another embodiment, step (e) is carried out in the presence of an amide coupling agent, a base, and solvent at a temperature from about 0° C. to about 100° C. Preferably, step (e) is carried out in the presence of 4-(4,6-Dimethoxy-1,3,5-triazin-2-yl)-4-methyl-morpholinium chloride, N-methylmorpholine, and DMF at a temperature from about 0° C. to about 100° C.

In another embodiment, steps (a) through (e) result in an overall stoichiometric yield of greater than 25% yield of the formula VI compound. Preferably, steps (a) through (e) result in an overall stoichiometric yield of greater than 45% yield of the formula VI compound.

In another aspect, the invention provides a compound of formula I, comprising 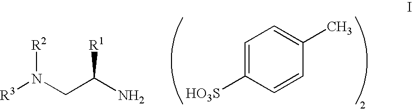
wherein:
R¹, R², and R³are independently, H, C₁-C₆ alkyl, 2-10 membered heteroalkyl, —(CR¹³R¹⁴)ₜ(C₆-C₁₀ aryl), —(CR¹³R¹⁴)ₜ(C₃-C₁₀ cycloalkyl), —(CR¹³R¹⁴)(C₆-C₁₀ heterocyclic), wherein t is an integer from 0 to 5; 1 or 2 ring carbon atoms of the cycloalkyl or heterocyclic group are optionally substituted with an oxo (═O) moiety; each R¹³ and R¹⁴ is independently H; C₁-C₆ alkyl, or 2-10 membered heteroalkyl, and wherein any of R¹, R² or R³ may be optionally substituted with one or more substituents independently selected from halo, —OH, —CN, —SR¹⁵, —NO₂, C₁-C₆ alkyl, C₂-C₆ alkenyl, C₂-C₆ alkynyl, 2-10 membered heteroalkyl, —COR¹⁵, or COOR¹⁵ wherein R¹⁵ is H, C₁-C₆ alkyl, or 2-10 membered heteroalkyl.

In one embodiment, R¹, R², and R³ are independently a C₁-C₆ alkyl, —(CR¹³R¹⁴)ₜ(C₆-C₁₀ aryl), —(CR¹³R¹⁴)(C₆-C₁₀ heterocyclic); unsubstituted or substituted with one or more substituents independently selected from the group consisting of C₁-C₆ alkyl and —O-alkyl. Preferably, R¹, R², and R³ are independently a C₁-C₆ alkyl group, unsubstituted or substituted with one or more substituents independently selected from the group consisting of C₁-C₆ alkyl and —O-alkyl. More preferably, R¹, R², and R³ are independently an unsubstituted C₁-C₃ alkyl group. More preferably, R¹, R², and R³ are each —CH₃.

In another aspect, the invention provides a compound of formula VII, comprising 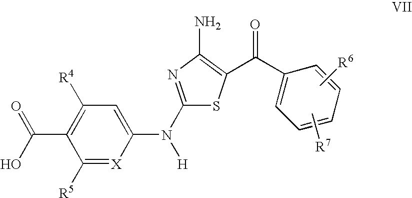
wherein:
R⁴ and R⁵ are independently H, halo, C₁-C₂ alkyl, —OCH₃, —OH, —NH₂, —NHCH₃, —N(CH₃)₂, —NO₂, —SH, —SCH₃, —S(O)CH₃, —SO₂CH₃,P(CH₃)₂, or PO₃H₂;
R⁶ and R⁷ are independently hydrogen, halo, methoxyl, or C₁-C₂ alkyl; and
X is —C— or —N—.

In one embodiment, R⁴ and R⁵ are independently H, halo, C₁-C₂ alkyl, —OCH₃, —OH; R⁶ and R⁷ are independently H, halo, methoxyl, or C₁₋₂ alkyl; and X is —C— or —N—. Preferably, R⁴ and R⁵ are independently H, halo, C₁-C₂ alkyl; R⁶ and R⁷ are independently H, halo, methoxyl, or C₁-C₂ alkyl; and X is —C— or —N—. More preferably, R⁴ and R⁵ are independently H or C₁-C₂ alkyl; R⁶ and R⁷ are independently H, halo, or C₁-C₂ alkyl; and X is —C— or —N—. More preferably, formula VII is the compound

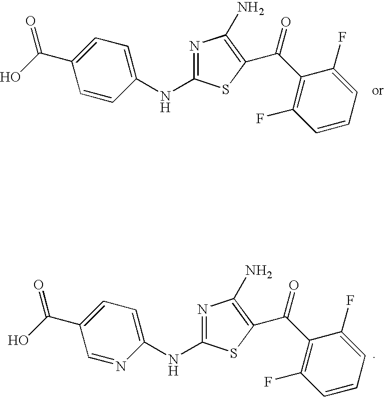

The terms “comprising” and “including” are used herein in their open, non-limiting sense.

The term “halo”, as used herein, unless otherwise indicated, means fluoro, chloro, bromo or iodo. Preferred halo groups are fluoro, chloro and bromo.

The term “alkyl”, as used herein, unless otherwise indicated, includes saturated monovalent hydrocarbon radicals having straight or branched moieties. A “C₁-C₆ alkyl” indicates a straight or branched alkyl moiety having 1 to 6 carbon atoms, and so forth.

The term “alkenyl” refers to a straight- or branched-chain alkenyl group having 2 to 12 carbon atoms in the chain. Illustrative alkenyl groups include prop-2-enyl, but-2-enyl, but-3-enyl, 2-methylprop-2-2nyl, hex-2-2nyl, ethenyl, pentenyl, and the like.

The term “alkynyl” refers to a straight- or branched-chain alkynyl group having from 2 to 12 carbon atoms in the chain. Illustrative alkynyl groups include prop-2-ynyl, but-2-ynyl, but-3-ynyl, 2-methylbut-2-ynyl, hex-2-ynyl, ethynyl, propynyl, pentynyl and the like.

The term “heteroalkyl” refers to a straight- or branched-chain alkyl group having from 2 to 10 atoms in the chain, one or more of which is a heteroatom selected from S, O, and N. Exemplary heteroalkyls include alkoxyls such as O-alkyl, alkyl ethers, secondary and tertiary alkyl amines, alkyl sulfides, and the like.

The term “cycloalkyl” refers to a saturated or partially saturated, monocyclic or fused or spiro polycyclic, carbocycle having from three to twelve ring atoms per ring. Illustrative examples of cycloalkyl groups include the following moieties:

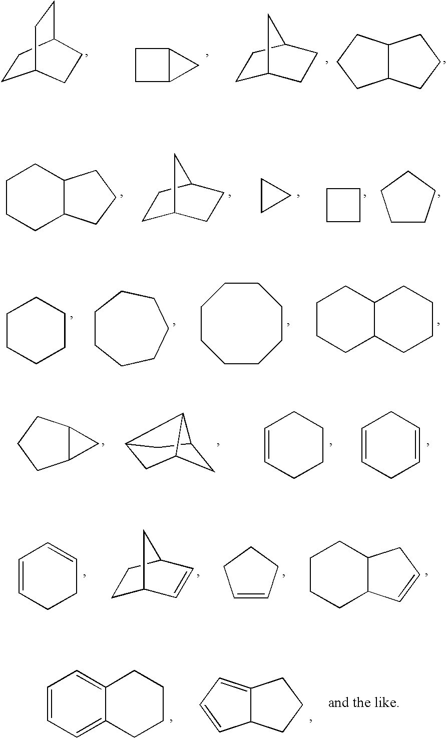

The term “aryl”, as used herein, unless otherwise indicated, includes an organic radical derived from an aromatic hydrocarbon by removal of one hydrogen, such as phenyl or naphthyl.

The term “4-10 membered heterocyclic”, as used herein, unless otherwise indicated, includes aromatic and non-aromatic heterocyclic groups containing one to four heteroatoms each selected from O, S and N, wherein each heterocyclic group has from 4-10 atoms in its ring system, and with the proviso that the ring of said group does not contain two adjacent O or S atoms. Non-aromatic heterocyclic groups include groups having only 4 atoms in their ring system, but aromatic heterocyclic groups must have at least 5 atoms in their ring system. The heterocyclic groups include benzo-fused ring systems. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). The 4-10 membered heterocyclic may be optionally substituted on any ring carbon, sulfur, or nitrogen atom(s) by one to two oxo, per ring. An example of a heterocyclic group wherein 2 ring carbon atoms are substituted with oxo moieties is 1,1-dioxo-thiomorpholinyl. Other Illustrative examples of 4-10 membered heterocyclic are derived from, but not limited to, the following:

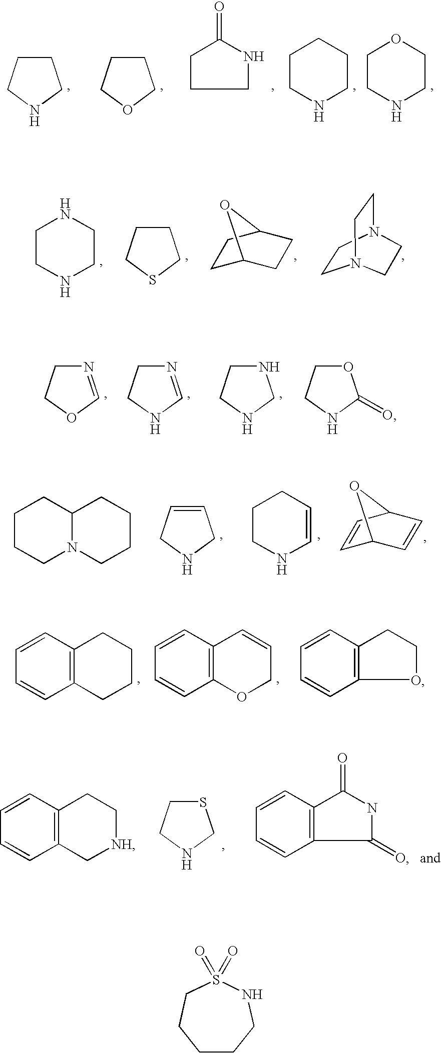

Unless otherwise indicated, the term “oxo” refers to ═O.

The term “amide” refers to the radical —C(O)N(R′)(R″) where R′ and R″ are each independently selected from hydrogen, alkyl, alkenyl, alkynyl, —OH, alkoxy, cycloalkyl, heterocycloalkyl, heteroaryl, aryl as defined above; or R′ and R″ cyclize together with the nitrogen to form a heterocycloalkyl or heteroaryl as defined above.

The term “substituted” means that the specified group or moiety bears one or more substituents. The term “unsubstituted” means that the specified group bears no substituents.

In accordance with a convention used in the art, 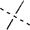
is used in structural formulae herein to depict the bond that is the point of attachment of the moiety or substituent to the core or backbone structure. Moreover, 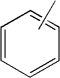
is used in structural formulae herein to depict that the point of attachment of the moiety or substituent to the core of the backbone aryl structure is unspecified. Where chiral carbons are included in chemical structures, unless a particular orientation is depicted, both stereoisomeric forms are intended to be encompassed. Certain compounds of formulae (I), (VI) and (VII) may have asymmetric centers and therefore exist in different enantiomeric forms. All optical isomers and stereoisomers of the compounds of formulae (I), (VI), and (VII), and mixtures thereof, are considered to be within the scope of the invention. With respect to the compounds of formulae (I), (VI) and (VII), the invention includes the use of a racemate, one or more enantiomeric forms, one or more diastereomeric forms, or mixtures thereof. The compounds of formulae (I), (VI) and (VII) may also exist as tautomers. This invention relates to the use of all such tautomers and mixtures thereof.

Certain functional groups contained within the compounds of the present invention can be substituted for bioisosteric groups, that is, groups which have similar spatial or electronic requirements to the parent group, but exhibit differing or improved physicochemical or other properties. Suitable examples are well known to those of skill in the art, and include, but are not limited to moieties described in Patini et al., Chem. Rev, 1996, 96, 3147-3176 and references cited therein.

The subject invention also includes isotopically-labelled compounds, which are identical to those recited in formulae (I), (VI) and (VII) but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., 3H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formulae (I), (VI) and (VII) of this invention and prodrugs thereof can generally be prepared by carrying out the procedures disclosed in the Schemes and/or in the Examples and Preparations below, by substituting a readily available isotopically labelled reagent for a non-isotopically labelled reagent.

Other aspects, advantages, and preferred features of the invention will become apparent from the detailed description below.

### DETAILED DESCRIPTION OF THE INVENTION AND ITS PREFERRED EMBODIMENTS

The present invention will be further illustrated in the following, non-limiting schemes and examples.

In the examples described below, unless otherwise indicated all temperatures are set forth in degrees Celsius (° C.) and all parts and percentages are by weight. Reagents were purchased from commercial suppliers such as Aldrich Chemical Company or Lancaster Synthesis Ltd. and were used without further purification unless otherwise indicated. Tetrahydrofuran and N,N-dimethylformamide were purchased from Aldrich in Sure Seal bottles and used as received. All solvents were purified using standard methods known to those skilled in the art, unless otherwise indicated.

The reactions set forth below were done generally under a positive pressure of argon at an ambient temperature (unless otherwise stated) in anhydrous solvents, and the reaction flasks were fitted with rubber septa for the introduction of substrates and reagents via syringe. Glassware was oven dried and/or heat dried. Analytical thin layer chromatography (TLC) was performed on glass-backed silica gel 60 F 254 plates from Analtech (0.25 mm), eluted with the appropriate solvent ratios (v/v), and were denoted where appropriate. The reactions were assayed by TLC and terminated as judged by the consumption of starting material.

Visualization of the TLC plates was done with iodine vapor, ultraviolet illumination, 2% Ce(NH₄)₄(SO₄)₄ in 20% aqueous sulfuric acid, phosphomolybdic acid/ethanol stain, or p-anisaldehyde spray reagent, and activated with heat where appropriate. Work-ups were typically done by doubling the reaction volume with the reaction solvent or extraction solvent and then washing with the indicated aqueous solutions using 25% by volume of the extraction volume unless otherwise indicated. Product solutions were dried over anhydrous Na₂SO₄ and/or Mg₂SO₄ prior to filtration and evaporation of the solvents under reduced pressure on a rotary evaporator and noted as solvents removed in vacuo. Flash column chromatography (Still et al., J. Org. Chem., 43, 2923 (1978)) was done using Merck silica gel (47-61 μm) with a silica gel crude material ratio of about 20:1 to 50:1, unless otherwise stated. Hydrogenolysis was done at the pressure indicated in the examples or at ambient pressure. All melting points (mp) are uncorrected.

¹H-NMR spectra were recorded on a Bruker or Varian instrument operating at 300 MHz and ¹³C-NMR spectra were recorded operating at 75 MHz. NMR spectra were obtained as CDCl₃ solutions (reported in ppm), using chloroform as the reference standard (7.27 ppm and 77.00 ppm), as DMSO-d₆ or CD₃OD (3.4 and 4.8 ppm and 49.3 ppm), or internally tetramethylsilane (0.00 ppm) when appropriate. Other NMR solvents were used as needed. When peak multiplicities are reported, the following abbreviations are used: s (singlet), d (doublet), t (triplet), q (quartet), m (multiplet), br (broadened), dd (doublet of doublets), dt (doublet of triplets). Coupling constants, when given, are reported in Hertz (Hz).

Infrared (IR) spectra were recorded on a Perkin-Elmer FT-IR Spectrometer as neat oils, as KBr pellets, or as CDCl₃ solutions, and when given are reported in wave numbers (cm⁻¹)

Mass spectrometry (MS) was conducted with various techniques. Mass spectra were obtained using liquid chromatograph electrospray ionization mass spectrometry, MS (ESP). Matrix-Assisted Laser Desorption/Ionization (MALDI) Fourier Transform Mass Spectrometry was performed on an IonSpec FTMS mass spectrometer.

The following process for preparing the chiral amine intermediates and 2,4-diaminothiazole amide compounds of the invention were made according to the general synthetic pathways shown in Schemes 1-4 and the detailed experimental procedures that follow. Where appropriate, the reactions were also assayed by HPLC. These synthetic pathways and experimental procedures utilize many common chemical abbreviations, such as MTBE (methyl t-butyl ether), THF (tetrahydrofuran), DMF (N,N-dimethylformamide), EtOAc (ethyl acetate), DBU (1,8-diazacyclo[5.4.0]undec-7-ene), EDC (1-(3-dimethylaminopropyl)-3-ethylcarbodiimide hydrochloride), HOBT (1-hydroxybenzotriazole hydrate), DMAP (4-dimethylaminopyridine), DIEA (diisopropylethylamine), and the like.

General Methods of Preparation

One aspect of this invention relates to a novel four-step process for preparing compounds of formula I, which are used as intermediates in the synthesis of 2,4-diaminothiazole amide compounds (VI). The four chemical steps are amide formation, amine deprotection, amide reduction, and salt formation. The last three of these four steps are carried out without the use of water as an extraction or chromatography purification agent, thereby enabling an efficient isolation of the water-soluble intermediates of formulae I and V. Accordingly, the formation of formula I compounds occurs under conditions that are nearly anhydrous, and thus loss of product is minimized. The corresponding bis-hydrochloride salts of formula I, as prepared in the literature, are extremely hygroscopic, leading to an overall loss of product.

As shown below in Scheme 1, the amides III result from a coupling of appropriate secondary amines and the D-chiral amines of formula II. Typical coupling reagents and corresponding conditions were employed, such as EDC, DCC, HOBt-H₂0, in CH₃CN or similar solvents. Examples of conventional procedures may be found in Isogai et al., J. Chem. Soc., Perkin Trans., 1, 1405-1411 (1984). The protecting group of the amide III is removed next to provide the deprotected amide IV, using standard reaction conditions, such as hydrogenation under reduced pressure in conjunction with a metal catalyst. See, e.g., Guthrie et al., J. Org. Chem., 46, 498-501 (1981). The deprotected amide IV can be used without further purification.

Conversion of the deprotected amide IV to chiral amine V is accomplished by reducing the amide group of IV using an appropriate hydride source (e.g., LiAlH₄, or a corresponding boron agent). This reaction may be carried out at a temperature from about 0° C. to about 100° C. in a suitable inert solvent, such as THF or other ethereal solvents. Preferably, the reaction is carried out at from about room temperature to about the reflux temperature of the solvent, for a time period ranging from about 5 minutes to 24 hours, preferably from about 10 to about 20 hours. The chiral amine V is isolated by first quenching the excess hydride reducing agent and then filtering off the by-product metal salts. Advantageously, the filtrate containing the highly water soluble chiral amine of V can be used in the next step without further purification.

The chiral amine V is then converted to the chiral amine bis-toluenesulfonic acid salt I. This conversion is conducted by adding p-toluenesulfonic acid hydrate to the filtrate of V, resulting in a white solid that was filtered to provide a total yield of about 45%. The reaction may be carried out at a temperature from about 0° C. to about 100 ° C. in a suitable inert solvent, such as THF. Preferably, the reaction is carried out at about room temperature for a time period ranging from about 5 minutes to 12 hours, preferably from about 1 to about 3 hours.

As also shown below in Scheme 1, structural amides VI described herein arose from straightforward amide formation via coupling of chiral amines I with the pendant acids on thiazoles VII. Typical coupling reagents and corresponding conditions were employed, such as EDC, HATU, pyBOP, 2-chloro-4,6-dimethoxy-1,3,5-triazine (Kaminski, Synthesis, 917-20 (1987)), 4-(4,6-dimethoxy-1,3,5-triazin-2-yl)-4-methylmorpholinium chloride (Kunishima et al., Tetrahedron, 55,13159-13170 (1999)), or many others that would be familiar to those skilled in the art.

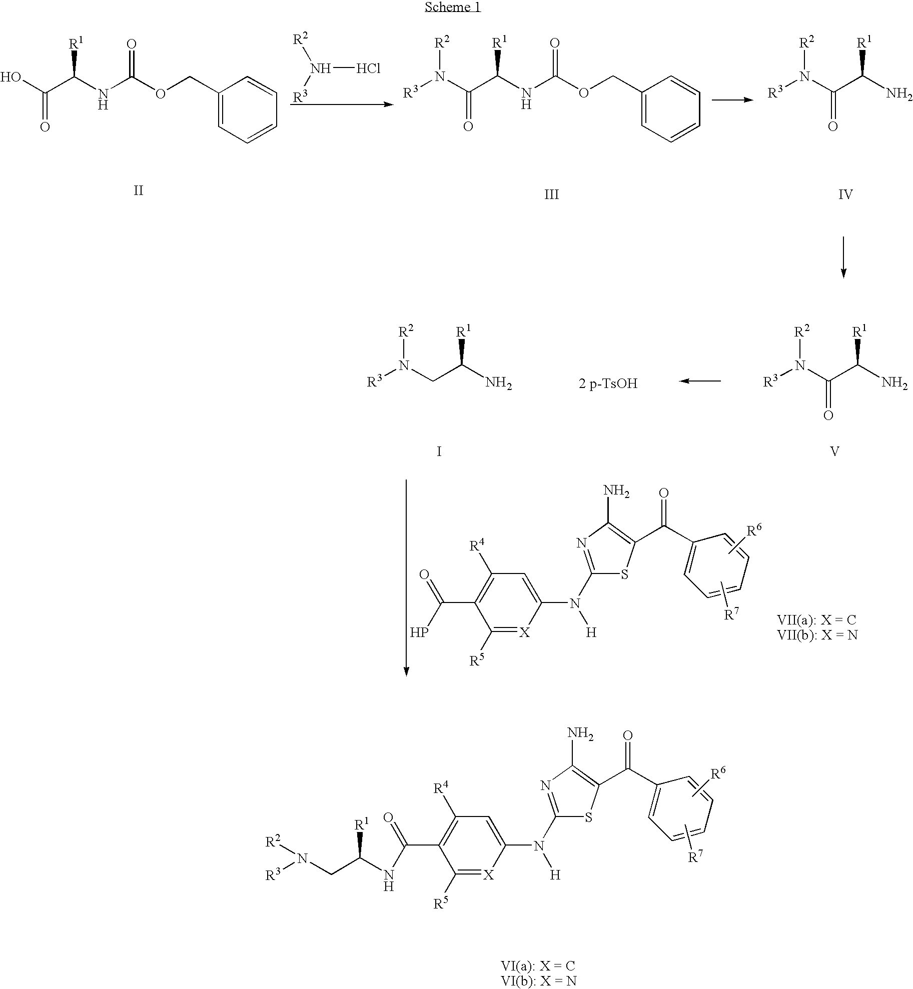

The acid coupling compounds of formula VII(a) are available as shown in synthetic Scheme 2 (when R⁴═R⁵═H and R⁶═R⁷═F) and other methods fully described in U.S. Provisional Patent Application No. 10/190,219, which is hereby incorporated in its entirety.

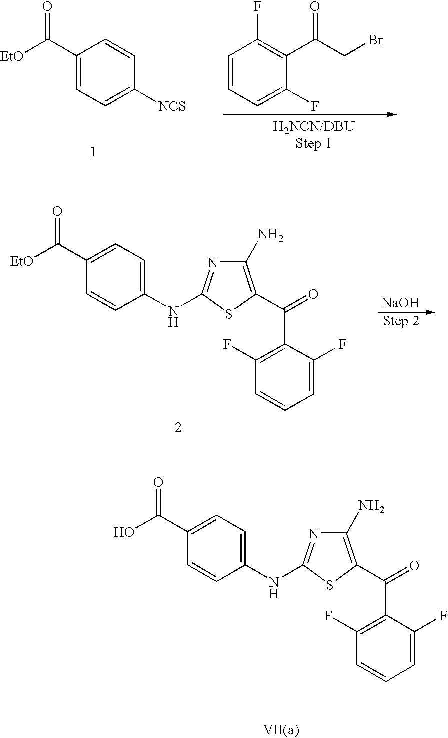

The acid coupling compounds of formula VII(b) are available as shown in synthetic Scheme 3 and other methods fully described in U.S. Provisional Patent Application No. 60/402,408, which is hereby incorporated in its entirety.

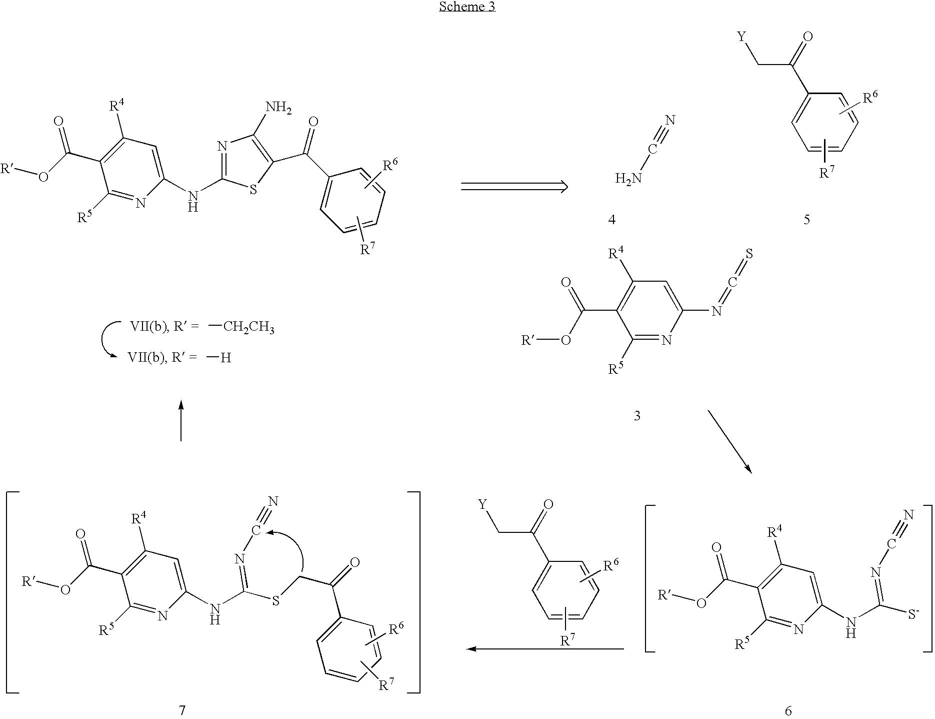

The requisite acids VII(b) were produced from saponification of the corresponding esters, typically ethyl esters (R′═—CH₂CH₃). As shown in Scheme 3, the convergence of three components culminates in the cyclization to the thiazole ring, as best exemplified in the literature by Gewald et al., J. Prakt. Chem., 35, 97-104 (1967) and International Publication No. WO 99/21845, which are hereby incorporated in their entireties. The initial condensation of isothiocyanates 3 and cyanamide 4 in the presence of a base, provides negatively charged isothiourea 6, which is treated in the same reaction vessel with α-haloacetophenone 5. The resultant S-alkyl-isothiourea 7 is deprotonated in the basic medium to effect cyclization to the thiazole VII(b). The bases employed can range from the simple alkoxides, as in the original Gewald protocol, to more hindered, non-nucleophilic bases such as potassium tert-butoxide or DBU that are more appropriate for the functionalities present in many of the examples. The starting components for the thiazoles, α-haloacetophenone 5, pyridyl-isothiocyanates 3, and its relatives, are available from multiple methods as described (in detail) in International Publication No. WO 99/21845. The corresponding amino-pyridines from the literature underwent treatment with thiophosgene, under either alkaline or acidic conditions as warranted.

As shown in Scheme 4 below, the isothiocyanates 3 for Scheme 3 were prepared upon conversion of corresponding amines 8 with traditional methods—typically thiophosgene, with either alkaline or acidic conditions as warranted, and as described in the prior citation WO 99/21845. Other methods can prepare isothiocyanates, such as treatment of amines with carbon disulfide in either acidic or alkaline media, or with hydrogen peroxide, see Li et al., J. Org. Chem., 62, 4539-4540 (1997) and references therein.

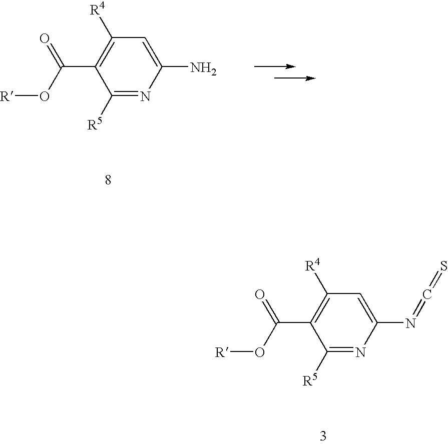

### EXAMPLE 1

### 1-Dimethylaminoprop-(2R)-yl-amine Bis-toluenesulfonic Acid Salt (10)

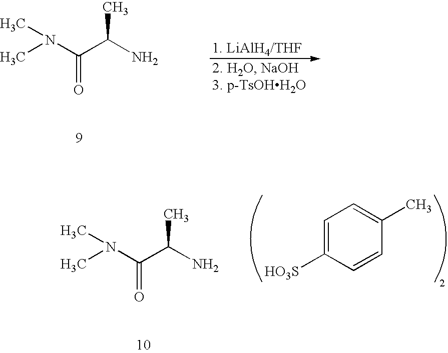

A LiAlH₄/THF solution (530 mL of a 1.0 M solution; 530 mmol) was added to a 3-L flask. D-Alanine dimethylamide (9) (29.2 9; 251 mmol) was dissolved in THF (1.00 L) to afford a cloudy, tan colored solution that was added dropwise to the LiAlH₄/THF solution over a period of 1 h. The resulting milky white suspension was heated at reflux for 17 h, and was then cooled slowly to ambient temperature. Water (20.1 mL) was added carefully to the reaction suspension over a period of 1.75 h. The resulting thick suspension was stirred at ambient temperature for 30 min. Sodium hydroxide (15% aqueous; 20.1 mL) was slowly added to the suspension over a period of 20 min and the suspension was allowed to stir at ambient temperature for 19 h. Water (60.3 mL) was added over a period of 10 min, and the mixture was stirred for 1 h at ambient temperature. The suspension was vacuum-filtered on paper and the white precipitate was washed with THF (250 mL).

In the next step, p-Toluenesulfonic acid (101 g; 528 mmol) was added to the filtrate and the resulting crystalline suspension was stirred at ambient temperature for 2 h. The suspension was concentrated to near dryness via vacuum distillation and the wet solid was azeotroped twice with absolute EtOH (2×400 mL). The wet solid was re-suspended in THF, vacuum-filtered, and the precipitate was then rinsed with THF (2×100 mL). The solid was suction dried briefly and was then dried in a vacuum oven at ˜60° C. for 24 h to afford 1-dimethylaminoprop-(2R)-yl-amine bis-toluenesulfonic acid salt (10) (77.9 g; 69.5%) as white crystals: ¹H NMR (300 MHz, MeOH-d₄) δ 7.72 (d, J=8.3, 4H), 7.25 (d, J=8.3 Hz, 4H), 3.88 (X portion of ABX, app sextet, J≈6.6 Hz, 1H), 3.49 (AB portion of ABX, J_{AX}=5.8 Hz, J_{BX}=2.2 Hz Δv=20.0 Hz, 2H), 2.96 (bs, 6H), 2.37 (s, 6H), 1.43 (d, J=6.6 Hz, 3H); ¹³C NMR (75 MHz, DMSO-d₆) δ 174.9, 45.8, 36.2, 35.0, 20.7; MS (Cl) m/z 103.1239 (103.1235 calcd for C₅H₁₅N₂, M+H⁺). IR (MeOH) 1216, 1177,1123,1011, 818, 687 cm⁻¹; elemental analysis calc for C₁₉H₃₀N₂O₆S₂: C, 51.10; H, 6.77; N, 6.27; 0,21.50; S, 14.36; found: C, 51.21; H, 6.72; N, 6.20; 0, 21.29; S, 14.26.

The starting materials for above were prepared as follows:
Step 1: N-Benzyloxycarbonyl-D-alanine Dimethylamide (11)

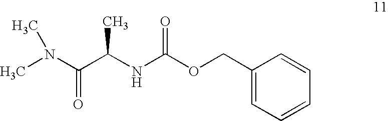

N-Benzyloxycarbonyl-D-alanine (10.4 g; 46.6 mmol) and HOBt-H₂O (6.30 g; 46.6 mmol) were suspended in CH₃CN (185 mL) at room temperature. The suspension was cooled to −3° C., and then a solution of dicyclohexylcarbodiimide (9.61 g; 46.6 mmol) in CH₃CN (21 mL) was added dropwise while maintaining the internal temperature below 2° C. After stirring at 0° C. for 2.5 h, (CH₃)₂NH—HCl (5.70 g; 69.9 mmol) was added. i-Pr₂NEt (24.3 mL; 140 mmol) was added dropwise while maintaining the internal temperature below 4° C. The suspension was stirred at 0° C. for 1.5 h, then was allowed to warm to room temperature and stir overnight (16 h). The suspension was concentrated to give a white solid, which was slurried in EtOAc (300 mL) and was then vacuum-filtered through a pad of Celite. The filtrate was washed with 1N HC1 (310 mL), 5% aqueous Na₂CO₃ (2×210 mL), saturated aqueous NaCl (105 mL), and was then dried over MgSO₄, filtered, and concentrated to give an oil/solid mixture. The residue was suspended in MTBE (210 mL) and was then stirred at room temperature for 24 h.

The suspension was vacuum-filtered through a pad of Celite, and the filtrate was concentrated to give N-benzyloxycarbonyl-D-alanine dimethylamide (9.3 g; 79%) as a slightly cloudy pale yellow oil and was used without any further purification. ¹H NMR (DMSO-d6); δ 7.43 (br d, 1H, J=7.7 Hz), 7.27-7.40 (m, 5H), 5.01 (s, 2H), 4.49 (app pentet, 1H, J=7.2 Hz), 3.00 (s, 3H), 2.82 (s, 3H), 1.16 (d, 3H, J=7.0 Hz). MS-APCl: m/z 251 (M+H⁺), 207 (M-NMe₂+H⁺), 117 (M-Cbz+2H⁺). ¹H NMR (CDCl₃) matched the literature spectrum (Isogai et al., J. Chem. Soc., Perkin Trans., 1, 1405-1411 (1984)).
Step 2: D-Alanine Dimethylamide (12)

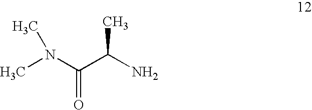

A mixture of N-benzyloxycarbonyl-D-alanine dimethylamide (78.7 g; 314 mmol), 10% palladium on carbon (7.87 g) and anhydrous ethanol (1.30 L) was hydrogenated with a Parr shaker (˜45 p.s.i. H₂). The mixture was vacuum-filtered through Celite, and the filtrate was concentrated to give D-alanine dimethylamide (30.4 g; 83%) as an oil contaminated with 4.5 mol % ethanol. This compound was used without further purification. ¹H NMR (DMSO-d₆): δ 3.83 (app q, 1H, J=6.8 Hz), 3.76 (br s, 2H), 2.97 (s, 3H), 2.81 (s, 3H), 1.09 (d, 3H, J=6.8 Hz); ¹³C NMR (DMSO-d6) δ 176.6, 47.5, 37.9, 36.8, 22.4; IR (MeOH) 1637,1374, 1258,1077 cm⁻¹; MS (Cl) m/z 117.1027 (117.1028 calcd for C₅H₁₃N₂O, M+H⁺). ¹H NMR (CDCl₃) matched the literature spectrum of DL-alanine dimethylamide (Guthrie et al., J. Org. Chem., 46, 498-501 (1981)).

### EXAMPLE 2

### 4-{Amino-[1-(2,6-difluorophenyl)-methanoyl]-thiazol-2-ylamino}-N-[2(R)-dimethylamino-1-methyl-ethyl]-benzamide (14)

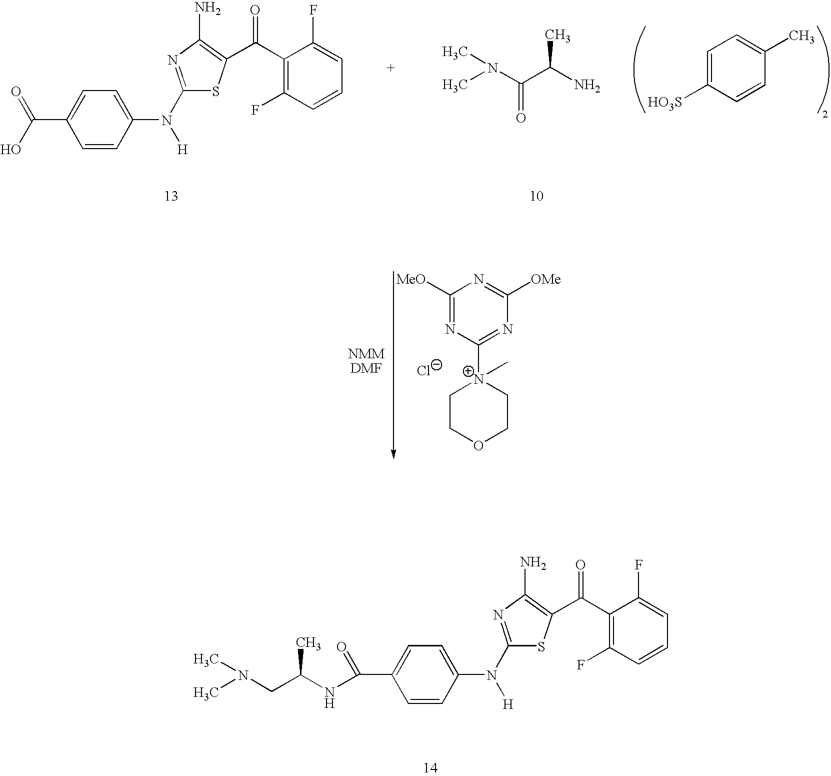

4-{4-Amino-5-[1-(2,6-difluorophenyl)-methanoyl]-thiazol-2-ylamino}-benzoic acid 13 (55.0 g; 147 mmol) and 1-dimethylaminoprop-2R-yl-amine bis-toluenesulfonic acid salt 10 (72.0 g; 161 mmol) were suspended in DMF (90 mL). N-methylmorpholine (NMM; 35.5 mL; 323 mmol) was added in one portion. 4-(4,6-Dimethoxy-1,3,5-triazin-2-yl)-4-methyl-morpholinium chloride (46.6 g; 168 mmol; see Kunishima, Tetrahedron, 55, 13159 (1999) for preparation) was added to the solution 1 h later. After stirring for 1 h, the solution was partitioned between EtOAc (1.10 L) and 5% aqueous Na₂CO₃ (1.00 L). The layers were separated and the organic layer was washed with 5% aqueous Na₂CO₃ (1.00 L). The organic layer was diluted with H₂O (1.00 L) and the resulting biphasic solution was acidified with CH₃CO₂H (15 mL) to pH 4.8. The layers were separated and the aqueous layer was washed with EtOAc (1.10 L). The organic layers were combined and discarded. The aqueous layer was diluted with EtOAc (1.10 L) and the resulting biphasic mixture was basified with 10% aqueous Na₂CO₃ (700 mL) to pH 9.7. The layers were separated, and the aqueous layer was extracted with EtOAc (1.10 L). The organic layers were combined, dried over MgSO₄, and were concentrated partially to afford a suspension. The suspension was vacuum-filtered and briefly suction-dried to afford crude 4-{amino-[1-(2,6-difluorophenyl)-methanoyl]-thiazol-2-ylamino}-N-[2(R)-dimethylamino-1-methyl-ethyl]-benzamide 14 (36.1 g; 53.6%) as a pale yellow solid. The crude product was then suspended in EtOH (180 mL). The suspension was heated to reflux, at which point all solids had dissolved. n-Heptane (180 mL) was added to the refluxing solution, which resulted in a crystal suspension that was stirred at reflux temperature for 45 min then cooled slowly to ambient temperature overnight. The suspension was vacuum-filtered to afford 4-{amino-[1-(2,6-difluorophenyl)-methanoyl]-thiazol-2-ylamino}-N-[2(R)-dimethylamino-1-methyl-ethyl]-benzamide (14) (31.4 9; 46.7%) as pale yellow crystals: m.p.=215-217° C.; ¹H NMR (500 MHz, DMSO-d6) δ 11.03 (brs, 1H), 8.29 (brs, 1H), 8.13 (br s, 1H), 8.03 (d, J=8.1 Hz, 1H), 7.83 (d, J=8.7 Hz, 2H), 7.64 (d, J=8.5 Hz, 2H), 7.51-7.57 (m, 1H), 7.18-7.23 (m, 2H), 4.13 (app septet, J=7.1 Hz, 1H), 2.37 (dd, J=7.6, 12.0 Hz, 1H), 2.20 (dd, J=6.9, 12.0 Hz, 1H), 2.15 (s, 6H), 1.12 (d, J=6.6 Hz, 3H); ¹³C NMR (125 MHz, DMSO-d₆) δ 172.9, 167.3, 164.8, 164.6, 158.2 (dd, J_{CF}=8.2, 247.5 Hz), 141.6, 131.6 (t, J_{CF}=9.8 Hz), 129.6, 128.3, 119.2 (t, J_{CF}=23.7 Hz), 118.2, 112.0-112.2 (m), 96.3, 64.3, 45.4, 43.0, 19.0; MS (Cl) m/z460.1615 (Exact Mass: 460.1619 calcd for C₂₂H₂₄F₂N₅O₂S, M +H⁺); elemental analysis calc for C₂₂H₂₃F₂N₅O₂S: C, 57.50; H, 5.05; F, 8.27; N, 15.24; O, 6.96; S, 6.98; found: C, 57.35; H, 5.03; F, 8.42; N, 15.27; O, 7.01; S, 6.96.

The 4-[4-Amino-5-(2,6-difluoro-benzoyl)-thiazol-2-ylamino]-benzoic acid (13) for the above was prepared as follows.
Step 1: 2-Bromo-2′,6′-difluoroacetophenone

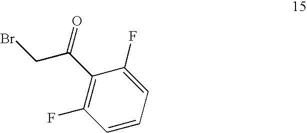

To a mechanically stirring solution of 2′,6′-difluoroacetophenone (15) (100.0 g, 640.0 mmol; Melford Laboratories, Ltd.) in ethyl acetate (1300 ml) was added freshly milled copper(II) bromide (300 g, 1.35 mol) and bromine (1.6 ml, 32 mmol). The mixture was heated at reflux for 2.25 hours and allowed to cool to room temperature. The resultant green mixture was filtered and the solids rinsed with ethyl acetate (4×100 ml). The filtrate was concentrated with a rotary evaporator at <40° C. under reduced pressure, diluted with methyl t-butyl ether (MTBE; 650 ml), filtered through a pad of silica gel (230-400μ; 9.5 cm diam.×4 cm. ht.), and solids rinsed with MTBE (5×200 ml). Concentration of the filtrate gave a pale green oil, which was purified by fractional vacuum distillation to give 117 g of pale yellow oil, bp 88-97° C. (2.0 mm Hg) in 78% yield. The results matched that previously described in International Publication No. WO 99/21845 (in Example C(79)) and was used without any further purification or characterization.

¹H NMR: δ 7.48 (ddd, ¹H, J=6.3, 8.5, 14.8 Hz), 7.01 (ddd, 2H, J=4.6, 5.8, 16.6 Hz), 4.37 (t, 2H, J=0.7 Hz).

Step 2: 4-{4-amino-5[1-(2,6-difluoro-benzoyl)]-thiazol-2-ylamino}-benzoic Acid Ethyl Ester (2)

To a mechanically stirring mixture of 4-ethoxycarbonylphenyl isothiocyanate (1, 105 g, 507 mmol; King's Research), cyanamide (22.3 g, 531 mmol), and acetonitrile (840 ml) at 1° C. was added dropwise over 30 minutes DBU (1,8-diazabicyclo [5.4.0]-7-undecene; 83.3 ml, 557 mmol). After 30 minutes at 0° C., a solution of 2-bromo-2′,6′-difluoro-acetophenone 15 (125 g, 532 mmol) in acetonitrile (125 ml) was added dropwise over 22 minutes. Upon warming to ambient temperature, the resultant mixture turned into a dark red solution, and was allowed to stir for 2 hours. Water (75 ml) was added dropwise, and the resultant yellow suspension stirred for 30 minutes. Additional water (925 ml) was added and the suspension was stirred overnight (19 h). The yellow solid was filtered, and was then rinsed with acetonitrile/water (1/1; 2×400 ml) and dried under high vacuum at 45° C. to give 193 g of yellow solid in 94% yield. MP=108-121° C.; ¹H NMR (DMSO-d₆) δ 11.16 (s, 1H), 8.22 (br s, 2H), 7.92-7.96 (m, 2H), 7.71-7.75 (m, 2H), 7.50-7.61 (m, 1H), 7.18-7.27 (m, 2H), 4.29 (q, J=7.1 Hz, 2H), 1.31 (t, J=7.1 Hz, 3H); ¹³C NMR (DMSO-d₆) δ 173.8, 167.5, 165.7, 165.0, 158.8 (dd, J_{CF}=8.1, 248.0 Hz), 143.9, 132.2 (t, J_{CF}=9.7 Hz), 130.9, 124.8, 119.7 (t, J_{CF}=23.6 Hz), 118.7, 112.5-112.7 (m), 97.2, 60.9, 14.6; IR (MeOH) 1718, 1621, 1529, 1467, 1432, 1278, 1258, 1177, 1108, 1004 cm⁻¹; MS (Cl) m/z 404.0871 (404.0880 calcd for C₁₉H₁₆F₂N₃O₃S, M+H⁺); elemental analysis calc for C₁₉H₁₅F₂N₃O₃S: C, 56.57; H, 3.75; N, 10.42; S, 7.95; F, 9.42; found: C, 56.44; H, 3.68; N, 10.33; S, 7.84; F, 9.42.

Step 3: 4-{4-amino-5[1-(2,6-difluoro-benzoyl)]-thiazol-2-ylamino}-benzoic Acid (13)

To a mechanically stirring solution of 4-{4-amino-5-[1-(2,6-difluoro-benzoyl)]-thiazol-2-ylamino}-benzoic acid ethyl ester (2, 80.0 g, 198 mmol) in ethanol (465 ml) was added 1.5N aqueous NaOH solution (264 ml). The mixture was heated at 70° C. overnight (20 h). While maintaining 70° C., the pH was slowly adjusted to 4.5 with 3N HCl (140 ml) to allow crystallization. 30 minutes later, the pH was further adjusted to 2.0 with 3N HCl (9 ml). The suspension was then allowed to cool to ambient temperature and stir overnight (18 h). The crystals were filtered and dried under vacuum at 45° C. to provide 70.7 g of canary yellow crystals in 95% yield. m.p. >250° C.; ¹H NMR (DMSO-d₆) δ 12.77 (s, 1H), 11.18 (s, 1H), 8.21 (br s, 2H), 7.98 (app d, J=8.7 Hz, 2H), 7.72 (app d, J=8.7 Hz, 2H), 7.49-7.61 (m, 1H), 7.17-7.26 (m, 2H); ¹³C NMR (DMSO-d₆) δ 173.6, 167.3, 167.1, 164.8, 158.6 (dd, J_{CF}=8.6, 247.7 Hz), 143.5, 132.1 (t, J_{CF}=10.5 Hz), 131.0, 125.5, 119.6 (t, J_{CF}=23.7 Hz), 118.5, 112.4-112.6 (m), 96.9; IR (MeOH) 3246, 1695, 1621, 1529, 1467, 1432, 1254, 1177, 1004 cm⁻¹; MS (Cl) m/z 376.0572 (376.0567 calcd for C₁₇H₁₂F₂N₃O₃S, M+H⁺); elemental analysis calc for C₁₇H₁₁F₂N₃O₃S·0.1 H₂O·0.05 NaCl: C, 53.72; H, 2.97; N, 11.06; S, 3.44; F, 10.00; Cl, 0.47; found: C, 54.02; H, 2.86; N, 11.09; S, 8.11; F, 9.96; Cl, 0.57.

### EXAMPLE 3

### 6-[4-Amino-5-(2,6-difluoro-benzoyl)-thiazol-2-ylamino]-N-(2-dimethylamino-1R-methyl-ethyl)-nicotinamide (16)

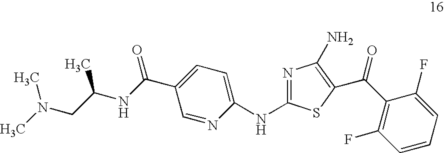

The title compound was prepared in a manner analogous to that used for Example 2. 1-Dimethylaminoprop-2R-yl-amine bis-toluenesulfonic acid salt and 6-[4-amino-5-(2,6-difluoro-benzoyl)-thiazol-2-ylamino]-nicotinic acid 17 provided a yellow solid in 70% yield. ¹H NMR (DMSO-d₆): δ 8.70 (d, 1H, J=2.1 Hz), 8.07 (d, 2H, J=8.3 Hz), 7.95-7.92 (m, 2H), 7.55-7.45 (m, 1H), 7.20 (dd, 1H, J=7.7, 8.1 Hz), 7.05 (d, 1H, J=8.7 Hz), 4.10-4.05 (m, 1H), 1.05 (d, 3H, J=6.6 Hz); LC-MS (M+H⁺): 461; Anal. Calcd. for C₂₁H₂₂F₂N₆O₂S.1.5 H₂O: C, 51.74; H, 5.17; N, 17.24; S, 6.58. Found: C, 52.04; H, 5.17; N, 17.29; S, 6.55.

The starting material for the 6-[4-amino-5-(2,6-difluoro-benzoyl)-thiazol-2-ylamino]-nicotinic acid 17 was available as follows:

The starting materials for above were prepared as follows:
Step 1: 6-Amino-nicotinic Acid Ethyl Ester (18)

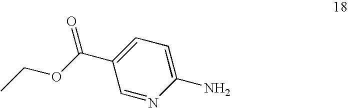

HCl gas was passed through a solution of 6-amino-nicotinic acid (2.00 g, 14.5 mmol) in ethanol (40 mL) at 0° C. for 10 min, then warmed to reflux. After 18 h, the mixture was allowed to cool, concentrated in vacuo to a colorless solid, which was partitioned with EtOAc and sat. aq. Na₂CO₃. The aqueous layer was separated and extracted with EtOAc. The combined organic layers were dried over Na₂SO₄ and evaporated. Recrystallization in EtOH gave 2.20 g (92% yield) of white solid that was typically used without further purification. ¹H NMR (CDCl₃): δ 8.75 (d, 1H, J=2.1 Hz), 8.07 (dd, 1H, J=8.7, 2.1 Hz), 6.51 (d, 1H, J=8.7 Hz), 4.96 (bs, 2H), 4.35 (q, 2H, J=7.2 Hz), 1.40 (t, 3H, J=7.2 Hz).
Step 2: 6-Isothiocyanato-nicotinic Acid Ethyl Ester (19)

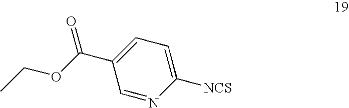

A solution of thiophosgene (1.10 mL, 14.4 mmol) in acetone (20 mL) and sat. aq. NaHCO₃ (40 mL) were both added simultaneously to a suspension of 6-amino-nicotinic acid ethyl ester (2.00 g, 12.0 mmol) in acetone (20 mL) and CHCl₃ (20 mL) at −10° C. (salt-ice bath at such a rate that the internal temperature did not exceed 5° C. Subsequently the acetone and chloroform were removed under reduced pressure. The resultant residue was extracted with CH₂Cl₂, concentrated, and purified with flash column using CH₂Cl₂ as eluant to give 1.54 g (61% yield) of orange crystals, which were used without any further purification. ¹H NMR (CDCl₃): δ 8.96 (d, 1H, J=2.2 Hz), 8.24 (dd, 1H, J=2.2, 8.3 Hz), 7.07 (d, 1H, J =8.3 Hz), 4.34 (q, 2H, J=7.2 Hz), 1.34 (t, 3H, J=7.2 Hz).
Step 3: 2-Bromo-2′,6′-difluoroacetophenone

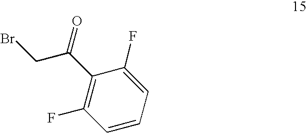

2-Bromo-2′,6′-difluoroacetophenone (15) was prepared according to the same procedure in Example 2.
Step 4: 2-[4-Amino-S(2, 6-difluoro-benzoyl)-thiazol-2-ylamino]-pyridine-5-carboxylic Acid Ethyl Ester (20)

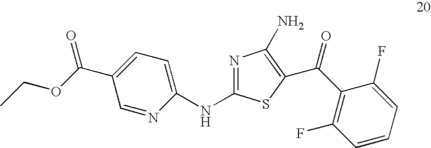

To a solution of 6-isothiocyanato-nicotinic acid ethyl ester (19, 300 mg, 1.44 mmol) in CH₃CN (5 mL) was added cyanamide (67 mg, 1.6 mmol) and 1,8-diazabicyclo[5.4.0]-undec-7-ene (DBU; 0.259 mL, 1.72 mmol). After 0.5 h, 2-bromo-2′,6′-difluoroacetophenone (15, 0.355 g, 1.51 mmol) and DBU (0.259 mL, 1.72 mmol) were added. After an additional 1.5 h, the solution was diluted with 10% MeOH/CHCl₃ (100 mL), washed with H₂O (25 mL×2), dried over Na₂SO₄, and concentrated to a brown solid, which was purified via column chromatography with 1% (58% NH₄OH) in 15% MeOH/CHCl₃ as eluant to give 161 mg (40% yield) of a brown powder, which was used without any further purification. ¹H NMR (DMSO-d₆): δ 8.78 (d, 1H, J=2.0 Hz), 8.21 (dd, 1H, J=2.2, 8.7 Hz), 8.00 (bs, 2H), 7.62-7.52 (m, 1H), 7.22 (dd, 2H, J=7.7, 8.1 Hz), 7.19 (d, 1H, J=8.8 Hz), 4.28 (q, 2H, J=7.1 Hz), 1.28 (t, 3H, J=7.1 Hz).
Step 5: 6-[4-Amino-5(2,6-difluoro-benzoyl)-thiazol-2-ylamino]-nicotinic Acid (17)

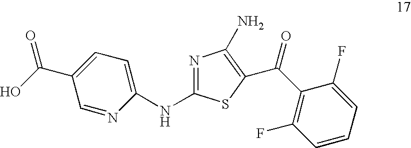

To a suspension of 6-[4-amino-5-(2,6-difluoro-benzoyl)-thiazol-2-ylamino]-nicotinic acid ethyl ester (0.29 g, 0.72 mmol) in MeOH (3 mL) was added 3N NaOH (2.4 mL, 7.2 mmol). The resulting solution was stirred at ambient temperature for 18 h. The methanol was removed in vacuo, and the resultant solution was brought to pH 3 with 10% aq. HCl. The precipitate was filtered off and dried to give 259 mg (96% yield) of a yellow solid, which was used without any further purification. ¹H NMR (DMSO-d₆): δ 12.37 (s, 1H), 8.75 (s, 1H), 8.02 (dd, 1H, J=2.1, 8.7 Hz), 8.01 (bs, 2H), 7.62-7.51 (m, 1H), 7.30-7.12 (m, 3H).

While the invention has been illustrated by reference to specific and preferred embodiments, those skilled in the art will recognize that variations and modifications may be made through routine experimentation and practice of the invention. Thus, the invention is intended not to be limited by the foregoing description, but to be defined by the appended claims and their equivalents.

1. A process for preparing a compound of formula I 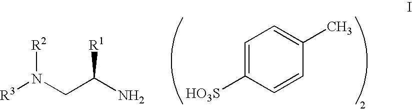
wherein:
R¹, R², and R³ are independently, H, C₁-C₆ alkyl, 2-10 membered heteroalkyl, —(CR¹³R¹⁴)ₜ(C₆-C₁₀ aryl), —(CR¹³R¹⁴)ₜ(C₃-C₁₀ cycloalkyl), —(CR¹³R¹⁴)(C₆-C₁₀ heterocyclic), wherein t is an integer from 0 to 5; 1 or 2 ring carbon atoms of the cycloalkyl or heterocyclic group are optionally substituted with an oxo (═O) moiety; each R¹³ and R¹⁴ is independently H, C₁-C₆ alkyl, or 2-10 membered heteroalkyl, and wherein any of R¹, R² or R³ may be optionally substituted with one or more substituents independently selected from halo, —OH, —CN, —SR¹⁵, —NO₂, C₁-C₆ alkyl, C₂-C₆ alkenyl, C₂-C₆ alkynyl, 2-10 membered heteroalkyl, —COR¹⁵, or COOR¹⁵ wherein R¹⁵ is H, C₁-C₆ alkyl, or 2-10 membered heteroalkyl;
comprising the steps of:
(a) coupling a compound of formula II 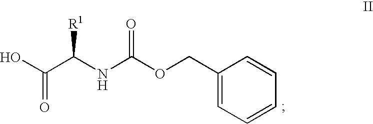
with an amine (R²)(R³)NH to form a compound of formula III 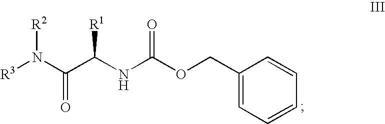
(b) deprotecting the compound of formula III to form the free amine compound of formula IV 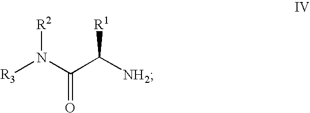
(c) reducing the free amine compound of formula IV to form a compound of formula V 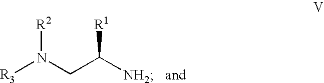
(d) treating the compound of formula V with p-toluenesulfonic acid to form the bis-toluenesulfonic acid salt compound of formula I;
wherein steps (b) and (c) can be reversed.

2. The process according to claim 1 wherein R¹, R², and R³ are independently a C₁-C₆ alkyl, —(CR¹³R¹⁴)ₜ(C₆-C₁₀ aryl), —(CR¹³R¹⁴)(C₆-C₁₀ heterocyclic), unsubstituted or substituted with one or more substituents independently selected from the group consisting of C₁-C₆ alkyl, and —O-alkyl.

3. The process according to claim 2 wherein R¹, R², and R³ are independently a C₁-C₆ alkyl group, unsubstituted or substituted with one or more substituents independently selected from the group consisting of C₁-C₆ alkyl and —O-alkyl

4. The process according to claim 3 wherein R¹, R², and R³ are independently an unsubstituted C₁-C₃ alkyl group.

5. The process according to claim 4 wherein R¹, R², and R³ are each —CH₃.

6. The process according to claim 1, where steps (b) through (d) are carried out without using water as a solvent or an extraction agent.

7. The process according to claim 1, wherein steps (c) and (d) are carried out without using water as a solvent or an extraction agent.

8. The process according to claim 1, wherein step (d) is carried out in the absence of water.

9. The process according to claim 1, where step (b) is carried out in the presence of hydrogen gas, a solvent, and a catalytic amount of metal catalyst, at a temperature from about 0° C. to about 100° C.

10. The process according to claim 1, wherein step (c) is carried out in the presence of a hydride source and a solvent at a temperature of from about 0° C. to about 100° C.

11. The process according to claim 10, wherein step (c) is carried out in the presence of lithium aluminum hydride in tetrahydrofuran at a temperature of from about 20° C. to about 70° C.

12. The process according to claim 1, wherein step (d) is carried out in the presence of tetrahydrofuran at a temperature from about 0° C. to about 70° C.

13. The process according to claim 1, wherein step (d) is carried out in the absence of an extraction or chromatography purification of the bis-toluenesulfonic acid salt compound of formula I.

14. The process according to claim 1, wherein steps (a) through (d) result in an overall stoichiometric yield of greater than 50% yield of the formula I compound.

15. The process according to claim 1, wherein steps (a) through (d) result in an overall stoichiometric yield of greater than 70% yield of the formula I compound.

16. The process for preparing a compound of formula VI 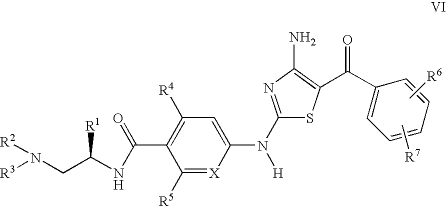
wherein:
R¹,R², and R³ are independently H, C₁-C₆ alkyl, 2-10 membered heteroalkyl, —(CR¹³R ⁴)ₜ(C₆-C₁₀ aryl), —(CR¹³R¹⁴)ₜ(C₃-C₁₀ cycloalkyl), —(CR¹³R¹⁴)(C₆-C₁₀ heterocyclic), wherein t is an integer from 0 to 5; 1 or 2 ring carbon atoms of the cycloalkyl or heterocyclic group are optionally substituted with an oxo (═O) moiety; each R¹³ and R¹⁴ is independently H, C₁-C₆ alkyl, or 2-10 membered heteroalkyl, and wherein any of R¹, R² or R³ may be optionally substituted with one or more substituents independently selected from halo, —OH, —CN, —SR¹⁵, —NO₂, C₁-C₆ alkyl, C₂-C₆ alkenyl, C₂-C₆ alkynyl, 2-10 membered heteroalkyl, —COR¹⁵, or COOR¹⁵ wherein R¹⁵ is H, C₁-C₆ alkyl, or 2-10 membered heteroalkyl;
R⁴ and R⁵ are independently H, halo, C₁₋₂ alkyl, —OCH₃, —OH, —NH₂, —NHCH₃, —N(CH₃)₂, —NO₂, —SH, —SCH₃, —S(O)CH₃, —SO₂CH₃,P(CH₃)₂, or PO₃H₂;
R⁶ and R⁷ are independently H, halo, methoxyl, or C₁₋₂ alkyl; and
X is —C— or —N—;
comprising the steps of:
(a) coupling a compound of formula II 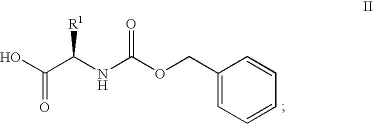
with an amine (R²)(R³)NH to form a compound of formula III 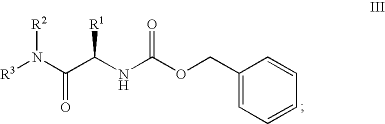
(b) deprotecting the compound of formula Ill to form the free amine compound of formula IV 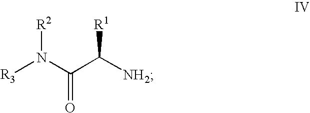
(c) reducing the free amine compound of formula IV to form a compound of formula V 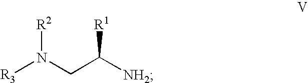
(d) treating the compound of formula V with p-toluenesulfonic acid hydrate to form the bis-toluenesulfonic acid salt compound of formula I 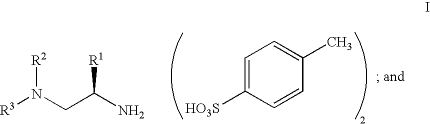
(e) coupling the bis-toluenesulfonic acid salt compound of formula I with a compound of formula VII 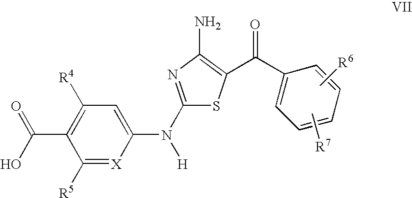
to form the compound of formula VI;
wherein steps (b) and (c) can be reversed.

17. A process according to claim 16 wherein R¹, R², and R³are independently a C₁-C₆ alkyl, 2-10 membered heteroalkyl, —(CR¹³R¹⁴)ₜ(C₆-C₁₀ aryl), —(CR¹³R¹⁴)(C₆-C¹⁰ heterocyclic), wherein t is an integer from 0 to 5; 1 or 2 ring carbon atoms of the cycloalkyl or heterocyclic group are optionally substituted with an oxo (═O) moiety; each R¹³ and R¹⁴ is independently H, C₁-C₆ alkyl, or 2-10 membered heteroalkyl, and wherein any of R¹, R² or R³ may be optionally substituted with one or more substituents independently selected from halo, —OH, —CN, —SR¹⁵, —NO₂, C₁-C₆ alkyl, C₂-C₆ alkenyl, C₂-C₆ alkynyl, 2-10 membered heteroalkyl, —COR¹⁵, or COOR¹⁵ wherein R¹⁵ is H, C₁-C₆ alkyl, or 2-10 membered heteroalkyl; R⁴ and R⁵ are independently H, halo, C₁₋₂ alkyl, —OCH₃, —OH, —NH₂, —NHCH₃, —N(CH₃)₂, —NO₂, —SH, —SCH₃, —S(O)CH₃, —SO₂CH₃,P(CH₃)₂, or PO₃H₂; R⁶ and R⁷ are independently H, halo, methoxyl, or C₁₋₂ alkyl; and X is —C— or —N—.

18. The process according to claim 17 wherein R¹, R², and R³are independently a C₁-C₆ alkyl group, unsubstituted or substituted with one or more substituents independently selected from the group consisting of C₁-C₃ alkyl and —O-alkyl; R⁴ and R⁵ are independently H, halo, C₁-C₂ alkyl, —OCH₃, —OH; R⁶and R⁷ are independently H, halo, methoxyl, or C₁-C₂ alkyl; and X is —C— or —N—.

19. The process according to claim 18 wherein R¹, R ², and R³are independently an unsubstituted C₁-C₃ alkyl group; R⁴and R⁵are independently H, halo, C₁-C₂ alkyl; R⁶and R⁷ are independently H, halo, methoxyl, or C₁-C₂ alkyl; and X is —C— or —N—.

20. The process according to claim 19 wherein R¹, R², and R³ are each —CH₃; R⁴and R⁵ are independently H or C₁-C₂ alkyl; R⁶and R⁷are independently H, halo, or C₁-C₂ alkyl; and X is —C— or —N—.

21. The process according to claim 16 wherein a compound of formula VI is selected from: 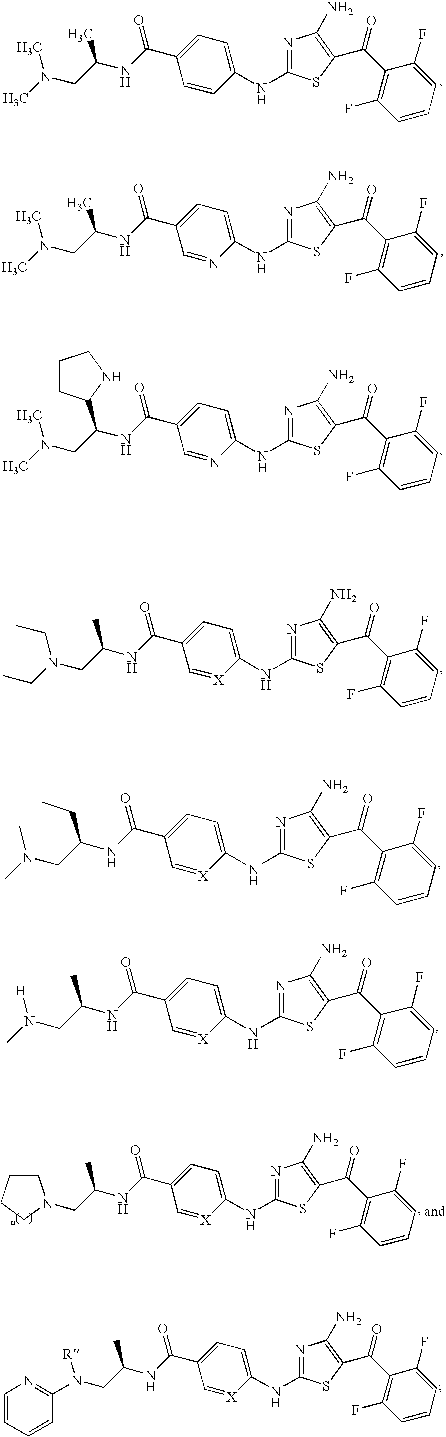
wherein n is 1 or 2 and R″ is H, —CH₃, or —CH₂CH₃.

22. The process according to claim 16, where steps (b) through (d) are carried out without using water as a solvent or an extraction agent.

23. The process according to claim 16, wherein steps (c) and (d) are carried out without using water as a solvent or an extraction agent.

24. The process according to claim 16, wherein step (d) is carried out in the absence of water.

25. The process according to claim 16, where step (b) is carried out in the presence of hydrogen gas, a solvent, and a catalytic amount of a metal catalyst, at a temperature from about 0° C. to about 100° C.

26. The process according to claim 16, wherein step (c) is carried out in the presence of a hydride source and a solvent at a temperature of from about 0° C. to about 100° C.

27. The process according to claim 26, wherein step (c) is carried out in the presence of lithium aluminum hydride in tetrahydrofuran at a temperature of from about 20° C. to about 70° C.

28. The process according to claim 16, wherein step (d) is carried out in the presence of tetrahydrofuran at a temperature from about 0° C. to about 70° C.

29. The process according to claim 16, wherein step (d) is carried out in the absence of an extraction or chromatography purification of the bis-toluenesulfonic acid salt compound of formula I.

30. The process according to claim 16, wherein step (e) is carried out in the presence of an amide coupling agent, a base, and solvent at a temperature from about 0° C. to about 100° C.

31. The process according to claim 30, wherein step (e) is carried out in the presence of 4-(4,6-Dimethoxy-1,3,5-triazin-2-yl)-4-methyl-morpholinium chloride, N-methylmorpholine, and DMF at a temperature from about 0° C. to about 100° C.

32. The process according to claim 16, wherein steps (a) through (e) result in an overall stoichiometric yield of greater than 25% yield of the formula VI compound.

33. The process according to claim 16, wherein steps (a) through (e) result in an overall stoichiometric yield of greater than 45% yield of the formula VI compound.

34. A compound of formula I, comprising 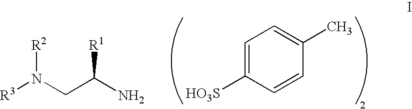
wherein:
R¹, R², and R³ are independently, H, C₁-C₈ alkyl, 2-10 membered heteroalkyl, —(CR¹³R¹⁴)ₜ(C₆-C₁₀ aryl), —(CR¹³R¹⁴)ₜ(C₃-C₁₀ cycloalkyl), —(CR¹³R¹⁴)(C₆-C₁₀ heterocyclic), wherein t is an integer from 0 to 5; 1 or 2 ring carbon atoms of the cycloalkyl or heterocyclic group are optionally substituted with an oxo (═O) moiety; each R¹³ and R¹⁴ is independently H, C₁-C₆ alkyl, or 2-10 membered heteroalkyl, and wherein any of R¹, R² or R³ may be optionally substituted with one or more substituents independently selected from halo, —OH, —CN, —SR¹⁵, —NO₂, C₁-C₆ alkyl, C₂-C₆ alkenyl, C₂-C₆ alkynyl, 2-10 membered heteroalkyl, —COR¹⁵, or COOR¹⁵ wherein R¹⁵ is H, C₁-C₆ alkyl, or 2-10 membered heteroalkyl.

35. The compound according to claim 34 wherein R¹, R², and R³ are independently a C₁₋₅ alkyl or monocyclic aryl or heteroaryl group, unsubstituted or substituted with one or more substituents independently selected from the group consisting of alkyl, heteroalkyl, and —O-alkyl.

36. The compound according to claim 35 wherein R¹, R², and R³are independently a C₁-C₆ alkyl group, unsubstituted or substituted with one or more substituents independently selected from the group consisting of C₁-C₆ alkyl and —O-alkyl.

37. The compound according to claim 36 wherein R¹, R², and R³ are independently an unsubstituted C₁-C₃ alkyl group.

38. The compound according to claim 37 wherein R¹, R², and R³ are each —CH₃.

39. A compound of formula VI, comprising 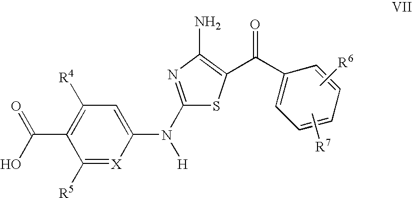
wherein:
R⁴ and R⁵ are independently H, halo, C₁₋₂ alkyl, —OCH₃, —OH, —NH₂, —NHCH₃, —N(CH₃)₂, —NO₂, —SH, —SCH₃, —S(O)CH₃, —SO₂CH₃,P(CH₃)₂, or PO₃H₂;
R⁶ and R⁷ are independently H, halo, methoxyl, or C₁₋₂ alkyl; and
X is —C— or —N—.

40. The compound according to claim 39 wherein R⁴ and R⁵ are independently H, halo, C₁-C₂ alkyl, —OCH₃, —OH; R⁶ and R⁷ are independently hydrogen, halo, methoxyl, or C₁-C₂ alkyl; and X is —C— or —N—.

41. The compound according to claim 40 wherein R⁴ and R⁵ are independently H, halo, C₁-C₂ alkyl; R⁶ and R⁷ are independently H, halo, methoxyl, or C₁-C₂ alkyl; and X is —C— or —N—.

42. The compound according to claim 40 wherein R⁴ and R⁵ are independently H or C₁₋₂ alkyl; R⁸ and R⁷ are independently H, halo, or C₁-C₂ alkyl; and X is —C— or —N—.

43. The compound according to claim 41 that is [chemical structure]
